(19) United States
(12) Patent Application Publication
(10) Pub. No.: US 2023/0381358 A1
(43) Pub. Date: Nov. 30, 2023

(54) Disinfectanator

(71) Applicant: Nina M. Riley, Boynton Beach, FL (US)

(72) Inventor: Nina M. Riley, Boynton Beach, FL (US)

(21) Appl. No.: 17/826,518

(22) Filed: May 27, 2022

(51) Int. Cl. A61L 2/22; A61L 9/14

(52) U.S. Cl. CPC A61L 2/22; A61L 9/14; A61L 2202/15; A61L 2202/25; A61L 2209/134

(57) ABSTRACT

Disinfectanator ensures complete disinfection of any enclosed space and all items inside the space. Disinfecting liquid in a portable canister releases via aerosol technology and creates disinfecting spray-mist. Gaseous nature of mist permeates entire space. Disinfectant travels through air to coat everything within the space. User releases spray-mist with click down lever on canister that locks into place. User places canister on flat surface and exits for product distribution. Upon completion, the space and all contents are sanitized and disinfected. Portable hand-held canister permits use in any space user can occupy. Travels to homes, vehicles, offices, vacations. Permeates into closets and cupboards, covers doorknobs, floors, walls, furniture and every surface, nook and cranny. No item or space remains untreated. Disinfectanator is quick, easy, effective. User enjoys peace of mind anywhere, anytime.

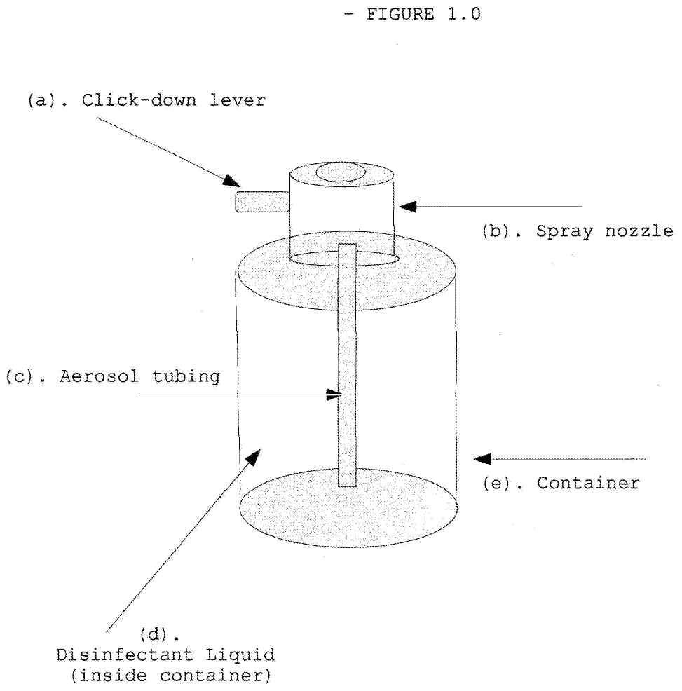

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION—FIELD OF INVENTION

This invention relates to disinfecting products and procedures. The invention provides a novel new and easier way to ensure that disinfection reaches all surfaces and hard to reach places without having to sanitize or wipe those surfaces by hand.

### BACKGROUND OF INVENTION

Unprecedented times have resulted in widespread concern for disinfecting and sanitizing surfaces and the spaces people occupy. Traveling and tourism has been affected because people are afraid that their hotel room or their house-sharing location has not been truly and properly sanitized.

People adapted by carrying small bottles of sanitizer and wipes with them everywhere. Large businesses or corporations can have a team come in with large sprayers that have to be carried around and aimed by a person to spray. However, for truly sanitizing a living, work, vehicle or vacation space, there is nothing available for the average consumer. This invention provides an easy way to effectively ensure that your space is sanitized.

Insecticide companies manufacture a small aerosol can with a click-down switch that consumers can easily use to release insecticide in their home, work or personal space. The aerosol mist travels through the entire space, leaving no surface, door handle, cupboard, nook or cranny untouched.

This invention is a new improvement on a existing mechanism in that it disperses disinfectant instead of insecticide.

The need for the invention is current and immediate. It can be used in personal spaces and also by hospitals or medical office. It will disinfect locker rooms, yoga studios, corporate offices, dorm rooms, personal trailers, tour and city buses and more. Any enclosed space will now be easy to sanitize and disinfect; allowing people to work, travel, vacation and even stay-at-home knowing that they have sanitized their space.

### BACKGROUND OF INVENTION—OBJECTS AND ADVANTAGES

(a) Ease of personal use: One object of the invention is to provide individual consumers within the general public an effective and simple method for full-coverage, easy to use disinfectant application in an aerosol container with a click-down switch to activate.

(b) Application reach: Another object of the invention is for disinfectant to permeate otherwise “hard-to-reach” spaces. For example, behind and all over heavy appliances or furniture, on ceilings and walls, in corners and crevices, inside confined spaces (for example cupboards, closets, underneath furniture), on carpeting, drapes, bedding, clothing, covering all surfaces such as tables, counters, chairs, doors and doorknobs.

(c) Recognition of need for product: while larger-scale solutions exist for disinfecting spaces, they are unaffordable, inaccessible and inconvenient for the general public and individual consumer. Rising concern and need for disinfecting spaces is part of daily life.

The invention addresses this need in home, work and travel locations. It ensures a consumer has the ability to disinfect any space quickly, conveniently and effectively.

(d) Ease of transport: An object of the invention is to provide a method of disinfectant that is portable and easy to transport, thereby facilitating use both inside and outside of the home; enabling the user to transport the invention to temporary housing, vacation homes, hotels, offices, dormitory rooms, automobiles, boats, recreational vehicles and to any space needing easy, effective, and full-coverage disinfectant.

The invention is the size of a hand-held canister, making it lightweight and easily transportable.

It can hold 200-300 ml of disinfecting product in the size of an insect-fogger aerosol can. A larger size similar to a can of spray paint can hold more product.

### SUMMARY

### Conclusion, Ramifications, and Scope

In conclusion, this invention is both an improvement on existing disinfecting procedures, and a novel method for distribution and application of disinfectant product.

The ramification is that people will be able to personally ensure that a space is sanitized before they occupy it, or before others occupy when they leave the space.

The scope is grand. Because the invention is small, lightweight and easy to use, it will travel well to any location. It can be sold everywhere from big box retailers to gas stations, camp stores, rental offices and more. Use does not have to be limited to individuals or small spaces. The invention can be sized to accommodate larger spaces.

The full scope of the invention is seen is the need for it.

### DRAWINGS-FIGURES BY REFERENCE LETTERS

(a). Shows the click-down lever. The lever clicks down and holds into place to release the product into the air via aerosol tubing mechanism and spray nozzle.

(b). Shows the spray nozzle. Disinfectant product is released through the spray nozzle into the air.

(c). Shows the aerosol tubing inside the container. Liquid travels up through the tubing into the spray nozzle for distribution via aerosol.

(d). Shows the disinfectant liquid inside the container. Disinfectant liquid is held inside the space of the container until aerosol release is activated by the click-down lever.

(e). Shows the container. Container is the receptacle for disinfectant liquid. It may be manufactured in various sizes to hold more or less disinfectant.

I claim:

1. The invention is a new and improved delivery system of disinfection and sanitization.

2. The invention is easy to use and application ensures full and complete coverage of any space and objects within the space. The consumer can “click it and forget it”. After the release of the disinfecting product into the air, the home, office or recreational space is clean and sanitized and ready to occupy within minutes. No mess, no spraying and wiping, no chance to miss a spot. It will provide sanitized spaces and peace of mind.

3. This invention can be quickly manufactured and affordably sold as it is an improvement on an existing mechanism. It is novel and unique in that is a new way for a private individual to evenly distribute disinfectant that will fully cover any and all surfaces that the aerosol mist reaches. It can be sold for under ten dollars.
